(19) United States
(12) Patent Application Publication
(10) Pub. No.: US 2021/0059851 A1
(43) Pub. Date: Mar. 4, 2021

(54) PLANTAR FASCIITIS SOLUTION

(71) Applicant: JOAN BROOKS, New Orleans, LA (US)

(72) Inventor: JOAN BROOKS, New Orleans, LA (US)

(21) Appl. No.: 16/551,288

(22) Filed: Aug. 26, 2019

(51) Int. Cl. A61F 5/14

(52) U.S. Cl. CPC A61F 5/14

(57) ABSTRACT

Invention titled Plantar Fasciitis Solution is a foot pad expressly designed to alleviate pain by strategically diverting pressure from painful areas of the foot of those suffering foot pain especially plantar fasciitis pain.

Invention is worn inside the shoe and is made of flexible vinyl material with moveable design projections to displace pressure and prevent pain areas from contacting hard surfaces while walking. Material is easily tailored by user to their foot size, fitting almost all types of shoes and boots.

Invention easy to clean, durable and travels well.

This invention is a foot pad specially designed to alleviate pain by distributing strategic pressure applied to the bottom of the foot, thus offering comfort to the pain sufferer.

The invention is a polyvinyl (ethylene, chemical based and/or rubber based) flexible pad in the shape of the foot-right and left styles. The size of the pad is determined by the size of the foot; purchaser may cut to size. The bottom of the pad can be smooth or have a gripping (non-slide) surface.

The (invention) pad will have flexible and moveable designed projections (on the top of the pad) towards the feet. The projection designs are varied and specific. The alignment and placement of the projections will vary in size, shape, flexibility, and number determined by the medical conditions of the foot and desired use of the pad (walking, comfort, sports, running). The invention will offer the additions of the arch supports and form supports for comfort and balance.

The heel area of the invention will serve the purposes of alleviating the pain of plantar fasciitis as well as various orthopedic conditions.

1. Invention Plantar Fasciitis Pain Solution provides pain relief to individuals suffering foot pain when walking especially those suffering from plantar fasciitis.
This invention will be a product made of vinyl material with several individual moveable flexible projections placement of which is specially designed for alleviation of pain.
Invention will be cut to individual foot size and is worn inside the shoe/boot. Invention will offer spongy cushion to prevent the wearer's foot especially in affected area from touching hard surface to aid in walking without pain.
Invention is comfortable and easy to use as well as durable and easy to clean.
Invention properties and benefits:
Instant foot pain relief
Provide comfort while walking
Reduce stress from pain
Diminishes incapacitation from painful walking
Made of vinyl with moveable flexible projections
Cuts to the size of the foot
Fosters a feeling of general wellbeing and healthfulness
Additionally, this product is easy to wash and is light for easy travel. This product will be an essential and extremely useful part of daily life for those suffering foot pain especially plantar fasciitis.
